(19) United States
(12) Patent Application Publication
(10) Pub. No.: US 2014/0005417 A1
(43) Pub. Date: Jan. 2, 2014

(54) WATER-INSOLUBLE COLORING COMPOUND, INK, RESIST COMPOSITION FOR COLOR FILTER, AND THERMAL TRANSFER RECORDING SHEET

(71) Applicants: Kaoru Takahashi, Saitama-shi (JP); Taichi Shintou, Saitama-shi (JP); Yutaka Tani, Yokohama-shi (JP); Masao Nakano, Kamakura-shi (JP); Takayuki Ujifusa, Ashigarakami-gun (JP); Satoshi Saito, Mishima-shi (JP); Yasuaki Murai, Kawasaki-shi (JP); Kei Inoue, Yokohama-shi (JP); Masashi Hirose, Machida-shi (JP); Takeshi Miyazaki, Ebina-shi (JP)

(72) Inventors: Kaoru Takahashi, Saitama-shi (JP); Taichi Shintou, Saitama-shi (JP); Yutaka Tani, Yokohama-shi (JP); Masao Nakano, Kamakura-shi (JP); Takayuki Ujifusa, Ashigarakami-gun (JP); Satoshi Saito, Mishima-shi (JP); Yasuaki Murai, Kawasaki-shi (JP); Kei Inoue, Yokohama-shi (JP); Masashi Hirose, Machida-shi (JP); Takeshi Miyazaki, Ebina-shi (JP)

(73) Assignee: CANON KABUSHIKI KAISHA, Tokyo (JP)

(21) Appl. No.: 14/005,312

(22) PCT Filed: Mar. 9, 2012
(86) PCT No.: PCT/JP2012/056793

§ 371 Date: Sep. 16, 2013

(30) Foreign Application Priority Data
Mar. 17, 2011 (JP) 2011-059555

(51) Int. Cl. C07D 311/82

(52) U.S. Cl. CPC C07D 311/82
USPC 549/394

(57) ABSTRACT

The present invention provides a water-insoluble coloring compound that has high solubility in solvent, good color tone and saturation, spectral reflectance characteristics for a wide color gamut, and a high light resistance. The present invention provides an ink containing the water-insoluble coloring compound. Furthermore, the present invention provides a resist composition for color filter and a thermal transfer recording sheet each produced using the ink.

The present invention provides a water-insoluble xanthene coloring compound having a specific structure.

### TECHNICAL FIELD

The present invention relates to a water-insoluble coloring compound and an ink containing the water-insoluble coloring compound, which is used in a process of producing, for example, a paint, an ink-jet ink, a color filter, or a resin molded product. The present invention also relates to a resist composition for color filter and a thermal transfer recording sheet each prepared using the ink.

### BACKGROUND ART

As methods for producing color filters, for example, a dyeing method, a printing method, an ink-jet method, and a photo-resist method are known. In particular, the photo-resist method can easily control the spectral characteristics in a reproducible manner and allows highly fine patterning because of its high resolution and is therefore a main method for producing color filters.

In the photo-resist method, the coloring agents are generally pigments. However, the pigment has a certain particle size and is thereby accompanied by a depolarization effect (collapse of polarization) to reduce the hiding ratio of color display of a liquid crystal display. Additionally, it is difficult to achieve a high transmission of backlight in a system using a pigment, which limits the improvement in brightness of a color filter. Furthermore, since the pigments are insoluble in organic solvents or polymers, color resist compositions thereof are obtained as dispersions of which stabilization is difficult.

In order to solve these problems in use of the pigments as coloring agents, a red color filter containing a xanthene dye, C.I. Solvent Red 49, has been reported (see PTL 1).

Incidentally, in a thermal transfer recording method, a thermal transfer sheet having a coloring material layer containing a heat-transferable coloring material and an image-receiving sheet having a coloring material-receiving layer on its surface are stacked on a sheet-like base material, and recording is performed by heating the thermal transfer sheet to transfer the coloring material in the thermal transfer sheet to the image-receiving sheet. In the thermal transfer recording method, the transfer sheet and the coloring material contained in the ink composition for the transfer sheet highly affect the transfer recording speed, the quality and the storage stability of the recorded image, etc. and are therefore very important. As a coloring material used in the thermal transfer recording method, an anthraquinone coloring material excellent in, for example, clearness, color reproducibility, and color optical density has been reported (see PTL 2).

### CITATION LIST

### Patent Literature

PTL 1 Japanese Patent Laid-Open No. 2003-344998

PTL 2 Japanese Patent Laid-Open No. 7-232481

### SUMMARY OF INVENTION

### Technical Problem

The color filter containing C.I. Solvent Red 49 described in PTL 1 does not have the depolarization effect and can provide high transmission of backlight, but has problems of insufficient light resistance and solubility in solvent. The thermal transfer recorded matter with the anthraquinone coloring material described in PTL 2 exhibits a satisfactory color tone, but the compatibility with light resistance cannot reach a satisfactory level.

Accordingly, the present invention provides solution to the above-described problems.

That is, the present invention provides a novel water-insoluble coloring compound that has a high affinity to a solvent, high brightness and saturation, spectral reflectance characteristics for a wide color gamut, and a high light resistance.

The present invention also provides an ink containing the water-insoluble coloring compound. Furthermore, the present invention provides a resist composition for color filter and a thermal transfer recording sheet each prepared using the ink.

### SOLUTION TO PROBLEM

The above-described problems are solved by the following invention.

The present invention relates to a water-insoluble coloring compound represented by Formula (1) shown below.

The present invention also relates to an ink containing at least a medium and a water-insoluble coloring compound represented by Formula (1):

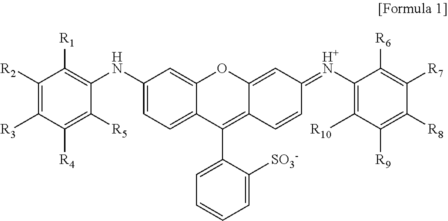

[wherein, R₁, R₅, R₆, and R₁₀ each independently represent an alkyl group; R₃ and R₈ each independently represent a hydrogen atom, an alkyl group, an alkoxy group, or an aryloxy group; and R₂, R₄, R₇, and R₉ each independently represent a hydrogen atom or an acylamino group represented by Formula (2), and at least one of R₂, R₄, R₇, and R₉ is an acylamino group represented by Formula (2):

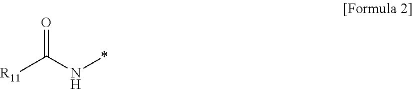

(wherein, R₁₁ represents an alkyl group, a cycloalkyl group, an aryl group, an arylalkyl group, an alkenyl group, or a heterocyclic group; and * represents a binding site)].

Furthermore, the present invention relates to a resist composition for color filter and a thermal transfer recording sheet each prepared using the ink.

### Advantageous Effects of Invention

The present invention can provide a novel water-insoluble coloring compound that has a high affinity to a solvent, good color tone and saturation, spectral reflectance characteristics for a wide color gamut, and a high light resistance, and can provide an ink containing the water-insoluble coloring compound. Furthermore, the present invention can provide a resist composition for color filter having a good color tone by using the ink. In addition, the present invention can provide a thermal transfer recording sheet having a good color tone by forming a coloring material layer of the ink on a base material.

### BRIEF DESCRIPTION OF DRAWINGS

FIG. 1 is a ¹H-NMR spectrum of a coloring compound (5) according to the present invention in DMSO-d₆ at 80° C. at 400 MHz.

FIG. 2 is a ¹H-NMR spectrum of a coloring compound (6) according to the present invention in DMSO-d₆ at 80° C. at 400 MHz.

FIG. 3 is a ¹H-NMR spectrum of a coloring compound (7) according to the present invention in DMSO-d₆ at 80° C. at 400 MHz.

FIG. 4 is a ¹H-NMR spectrum of a coloring compound (8) according to the present invention in DMSO-d₆ at 80° C. at 400 MHz.

FIG. 5 is a ¹H-NMR spectrum of a coloring compound (10) according to the present invention in DMSO-d₆ at 80° C. at 400 MHz.

FIG. 6 is a ¹H-NMR spectrum of a coloring compound (24) according to the present invention in DMSO-d₆ at 80° C. at 400 MHz.

FIG. 7 is a ¹H-NMR spectrum of a coloring compound (25) according to the present invention in DMSO-d₆ at 80° C. at 400 MHz.

### DESCRIPTION OF EMBODIMENTS

The present invention will now be described in more detail by preferred embodiments.

The present inventors have diligently studied for solving the above-mentioned problems and, as a result, have found that a water-insoluble coloring compound represented by Formula (1) shown below has a high affinity to a solvent, good color tone and saturation, spectral reflectance characteristics for a wide color gamut, and a high discoloration resistance,

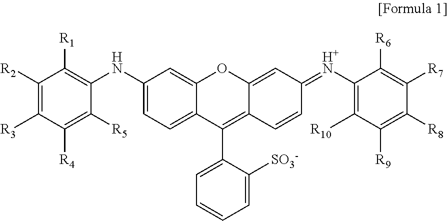

[wherein, R₁, R₅, R₆, and R₁₀ each independently represent an alkyl group; R₃ and R₈ each independently represent a hydrogen atom, an alkyl group, an alkoxy group, or an aryloxy group; and R₂, R₄, R₇, and R₉ each independently represent a hydrogen atom or an acylamino group represented by the following Formula (2), and at least one of R₂, R₄, R₇, and R₉ is an acylamino group represented by the following Formula (2):

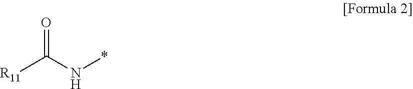

(wherein, R₁₁ represents an alkyl group, a cycloalkyl group, an aryl group, an arylalkyl group, an alkenyl group, or a heterocyclic group; and * represents a binding site)].

The present inventors have also found that a resist composition for color filter and a thermal transfer recording sheet each having a good color tone can be obtained by using an ink containing the water-insoluble coloring compound and have accomplished the present invention.

The water-insoluble coloring compound represented by Formula (1) will be described.

The water-insoluble coloring compound represented by Formula (1) of the present invention has a high affinity to an organic solvent. In the present invention, the term “water-insoluble” refers to that the solubility in water is less than 1% as mass percentage.

In Formula (1), examples of the alkyl group as R₁, R₅, R₆, or R₁₀ include a methyl group, an ethyl group, a n-propyl group, an isopropyl group, a n-butyl group, an isobutyl group, a sec-butyl group, and a tert-butyl group.

In Formula (1), the alkyl group as R₁, R₅, R₆, or R₁₀ may be further substituted with a substituent. Examples of the substituent include alkoxy groups, cyano groups, and halogen atoms. Specific examples of the alkyl group having a substituent as R₁, R₅, R₆, or R₁₀ include a hydroxyethyl group, a methoxyethyl group, a cyanoethyl group, and a trifluoromethyl group.

In Formula (1), R₁, R₅, R₆, and R₁₀ can be appropriately selected from the above-mentioned functional groups, and, from the viewpoint of ease of production, R₁ and R₆ may be the same, and R₅ and R₁₀ may be the same. Furthermore, these functional groups may be methyl groups from the viewpoint of obtaining raw materials.

In Formula (1), examples of the alkyl group as R₃ or R₈ include a methyl group, an ethyl group, a n-propyl group, an isopropyl group, a n-butyl group, an isobutyl group, a sec-butyl group, and tert-butyl group.

In Formula (1), examples of the alkoxy group as R₃ or R₈ include a methoxy group, an ethoxy group, and an isopropoxy group.

In Formula (1), examples of the aryloxy group as R₃ or R₈ include a phenoxy group and a naphthoxy group.

In Formula (1), the alkyl group, the alkoxy group, and the aryloxy group as R₃ or R₈ may be further substituted with substituents. Examples of the substituent include alkyl groups, aryl groups, arylalkyl groups, hydroxyl groups, carbamoyl groups, sulfamoyl groups, alkoxy groups, cyano groups, and halogen atoms. Specific examples of the alkyl, alkoxy, or aryloxy group further having a substituent as R₃ or R₈ include a hydroxyethyl group, a methoxyethyl group, a cyanoethyl group, a trifluoromethyl group, a methoxyethoxy group, a hydroxyethoxy group, a p-methoxyphenoxy group, an o-methoxy-phenoxy group, a tolyloxy group, and a xylyloxy group.

In Formula (1), R₃ and R₈ can be appropriately selected from the above-mentioned substituents and hydrogen atoms, and, from the viewpoint of light resistance, may be each a methyl group, an ethyl group, or a propyl group. When R₃ and R₈ are substituents other than hydrogen atoms, the substituents may be the same from the viewpoint of ease of production.

In Formula (1), R₂, R₄, R₇, and R₉ each independently represent a hydrogen atom or an acylamino group represented by Formula (2), and at least one of R₂, R₄, R₇, and R₉ is an acylamino group represented by Formula (2). In order that the water-insoluble coloring compound of Formula (1) has both a high color developing property and a high light resistance, at least one of R₂, R₄, R₇, and R₉ must be an acylamino group represented by Formula (2).

In particular, from the viewpoint of further enhancing the color developing property and the light resistance, two to four of the R₂, R₄, R₇, and R₉ in Formula (1) should be acylamino groups represented by Formula (2). In such a case, from the viewpoint of ease of production, the acylamino groups may be the same and, in Formula (1), R₂ and R₇ may be the same, and R₄ and R₉ may be the same.

In Formula (2), examples of the alkyl group as R₁₁ include a methyl group, an ethyl group, a n-propyl group, an isopropyl group, a n-butyl group, an isobutyl group, a sec-butyl group, and a tert-butyl group.

In Formula (2), examples of the cycloalkyl group as R₁₁ include a cyclopentyl group, a cyclohexyl group, and a cycloheptyl group.

In Formula (2), examples of the aryl group as R₁₁ include a phenyl group.

In Formula (2), examples of the arylalkyl group as R₁₁ include a benzyl group and a 2-phenethyl group.

In Formula (2), examples of the alkenyl group as R₁₁ include a vinyl group, a 1-propenyl group, a 2-propenyl group, a 1-methylethenyl group, a 1-butenyl group, a 2-butenyl group, and a 3-butenyl group.

In Formula (2), examples of the heterocyclic group as R₁₁ include an imidazolyl group, a benzoimidazolyl group, a pyrazolyl group, a benzopyrazolyl group, a triazolyl group, a thiazolyl group, a benzothiazolyl group, an isothiazolyl group, a benzoisothiazolyl group, an oxazolyl group, a benzooxazolyl group, a thiadiazolyl group, a pyrrolyl group, a benzopyrrolyl group, an indolyl group, an isoxazolyl group, a benzoisoxazolyl group, a thienyl group, a benzothienyl group, a furyl group, a benzofuryl group, a pyridyl group, a quinolyl group, an isoquinolyl group, a pyridazinyl group, a pyrimydinyl group, a pyrazinyl group, a cinnolinyl group, a phthalazinyl group, a quinazolinyl group, a quinoxalinyl group, and a triazinyl group.

In Formula (2), each group as R₁₁ may be further substituted with a substituent. Examples of the substituent include alkyl groups, aryl groups, arylalkyl groups, alkenyl groups, alkoxy groups, cyano groups, alkylamino groups, sulfoalkyl groups, carbamoyl groups, sulfamoyl groups, sulfonylamino groups, and halogen atoms. Specific examples of the group having a substituent as R₁₁ include a hydroxyethyl group, a methoxyethyl group, a cyanoethyl group, a trifluoromethyl group, a p-tolyl group, a p-methoxyphenyl group, and an o-chlorophenyl group.

In Formula (2), R₁₁ can be appropriately selected from the above-mentioned substituents. R₁₁ may be an alkyl group, a cycloalkyl group, an aryl group, or an arylalkyl group from the viewpoint of color developing property, and R₁₁ may be an alkyl group or an aryl group from the viewpoint of ease of production. In particular, when R₁₁ is a linear alkyl group, a higher light resistance can be obtained.

As shown in the following drawing, the water-insoluble coloring compound represented by Formula (1) of the present invention includes tautomers represented by, for example, the following Formulae (3) and (4), and these tautomers are included in the scope of the present invention.

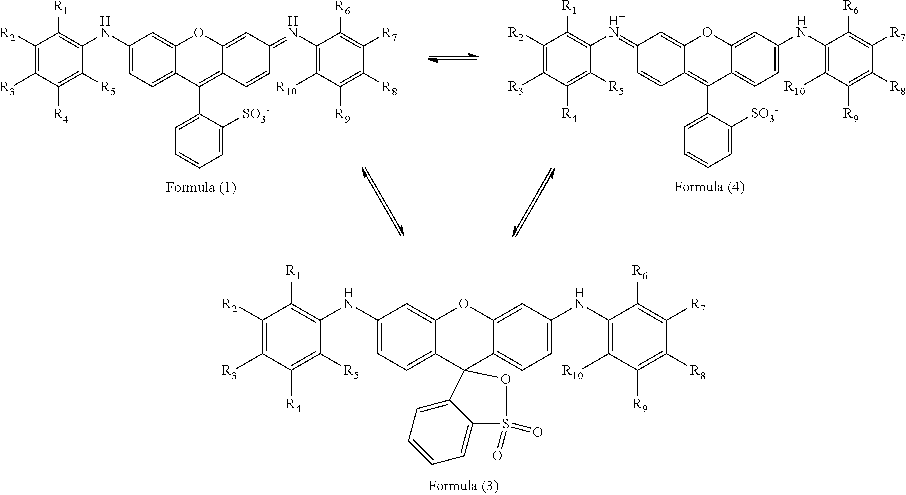

[R₁ to R₁₀ in Formulae (3) and (4) are synonymous with R₁ to R₁₀ in Formula (1)].

The water-insoluble coloring compound represented by Formula (1) of the present invention can be synthesized based on a known method. An example of the synthesis scheme is shown below:

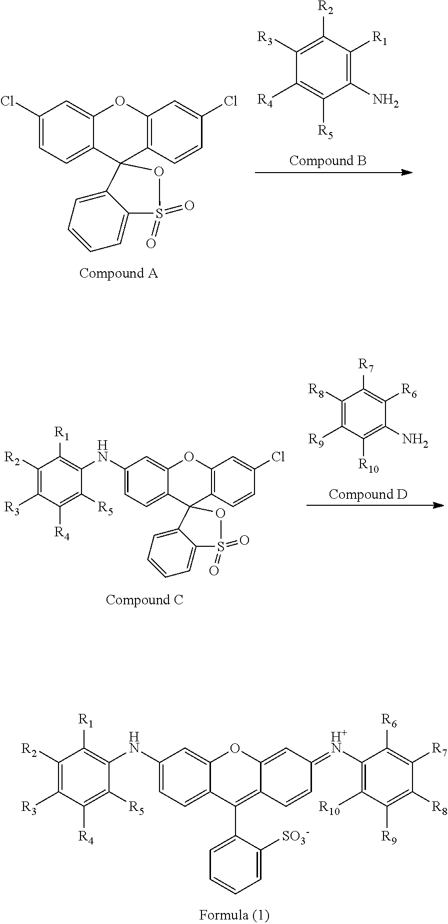

[R₁ to R₁₀ in Compounds B, C, and D are synonymous with R₁ to R₁₀ in Formula (1)].

In the scheme exemplified above, the water-insoluble coloring compound represented by Formula (1) is synthesized by a first condensation step shown in the first stage and a second condensation step shown in the second stage.

In the first condensation step, Compound A and Compound B are condensed by heating them in the presence or absence of an organic solvent and a condensing agent to synthesize Compound C. Subsequently, as shown in the second stage, Compound C prepared in the first condensation step and Compound D are heat-condensed to obtain a water-insoluble coloring compound represented by Formula (1).

Compound A and aniline derivatives serving as Compounds B and C are marketed and easily available. They also can be easily synthesized by known methods.

The organic solvent used in the condensation reaction in the synthesis scheme exemplified above will be described. Any organic solvent that does not participate in the reaction can be used in this step. Examples of the organic solvent include, but not limited to, methanol, ethanol, n-propanol, isopropanol, n-butanol, toluene, xylene, ethylene glycol, N-methylpyrrolidone, N,N-dimethylacetamide, N,N-dimethylformamide, dimethyl sulfoxide, sulfolane, chlorobenzene, dichlorobenzene, trichlorobenzene, and nitrobenzene. These solvents can be used alone or in combination of two or more thereof, depending on solubility of a substrate.

Examples of the condensing agent used in this step include magnesium oxide, zinc chloride, and aluminum chloride.

This step is usually performed at a temperature range of 60 to 220° C. and is usually completed within 24 hours.

The reaction temperature in the first condensation reaction may be in a range of 60 to 100° C. such as a range of 70 to 90° C. In such a range, the reaction rate can be appropriately controlled to inhibit excessive reaction and allows easy purification. The reaction temperature in the second condensation may be in a range of 120 to 220° C. such as a range of 180° C. or less. In such a range, the reaction rate can be appropriately controlled to satisfactorily prevent degradation of the generated compound.

In the case of synthesizing a water-insoluble coloring compound where the substituents R_{I}, R₂, R₃, R₄, and R₅ in Formula (1) are the same as the substituents R₆, R₇, R₈, R₉, and R₁₀, respectively, Compound B and Compound D in the above-mentioned scheme can be the same compound. Accordingly, in such a case, the water-insoluble coloring compound represented by Formula (1) can be obtained from Compound A by a single condensation step. Such a step is usually performed at a reaction temperature range of 100 to 220° C. and is usually completed within 24 hours.

The final product prepared by the above-mentioned reaction scheme can be obtained as a high-purity coloring compound by performing post-treatment in ordinary organic synthesis and then purification such as recrystallization, reprecipitation, or column chromatography. The water-insoluble coloring compound represented by Formula (1) can be identified by, for example, ¹H nuclear magnetic resonance (NMR) spectrometric analysis, liquid chromatography-time of flight mass spectrometry (LC/TOF MS), or UV/Vis spectrophotometry.

The water-insoluble coloring compound represented by Formula (1) can be used alone or optionally in combination of two or more thereof or may be used in combination with a known magenta pigment or dye, as a coloring agent. The water-insoluble coloring compound represented by Formula (1) can also be used as a lake pigment.

The ink of the present invention will now be described.

The water-insoluble coloring compound represented by Formula (1) of the present invention has a high affinity to an organic solvent, high brightness and saturation, spectral reflectance characteristics for a wide color gamut, and a high light resistance. Accordingly, the water-insoluble coloring compound is suitable as a coloring agent of an ink.

The ink of the present invention contains at least a medium and a water-insoluble coloring compound represented by Formula (1).

In the ink of the present invention, constitutional components other than the above-mentioned components are each determined depending on the intended use of the ink, and the ink can contain additives within ranges that do not impair the characteristics necessary in the use of the ink.

The ink of the present invention can be used as an ink-jet ink and also as, for example, a printing ink, a paint, or a writing material ink. In particular, the ink of the present invention can be suitably used as an ink for a red resist for color filter described below or as an ink for a thermal transfer recording sheet.

The ink of the present invention can be obtained, for example, as follows.

A water-insoluble coloring compound of the present invention and optional other additives such as a coloring agent, an emulsifier, and a resin are gradually added to a medium while stirring for mixing them thoroughly and evenly. Furthermore, the water-insoluble coloring compound is stably dissolved or fine-dispersed by applying a mechanical shear force with a dispersing machine to obtain an ink of the present invention.

In the present invention, the term “medium” refers to water or an organic solvent.

In the case of using an organic solvent as the medium of the ink of the present invention, the type of the organic solvent is determined depending on the intended use of the coloring agent and is not particularly limited. Examples of the organic solvent include alcohols such as methanol, ethanol, denatured ethanol, isopropanol, n-butanol, isobutanol, tert-butanol, sec-butanol, 2-methyl-2-butanol, 3-pentanol, octanol, benzyl alcohol, and cyclohexanol; glycols such as methyl cellosolve, ethyl cellosolve, diethylene glycol, and diethylene glycol monobutyl ether; ketones such as acetone, methyl ethyl ketone, and methyl isobutyl ketone; esters such as ethyl acetate, butyl acetate, ethyl propionate, and cellosolve acetate; aliphatic hydrocarbons such as hexane, octane, petroleum ether, and cyclohexane; aromatic hydrocarbons such as benzene, toluene, and xylene; halogenated hydrocarbons such as carbon tetrachloride, trichloroethylene, and tetrabromoethane; ethers such as diethyl ether, dimethyl glycol, trioxane, and tetrahydrofuran; acetals such as methylal and diethyl acetal; organic acids such as formic acid, acetic acid, and propionic acid; and sulfur/nitrogen-containing organic compounds such as nitrobenzene, dimethylamine, monoethanolamine, pyridine, dimethyl sulfoxide, and dimethylformamide.

The organic solvent that can be used in the ink of the present invention may be a polymerizable monomer. The polymerizable monomer can be an addition polymerizable or condensation polymerizable monomer and may be an addition polymerizable monomer. Examples of the polymerizable monomer include styrene monomers such as styrene, α-methylstyrene, α-ethylstyrene, o-methylstyrene, m-methylstyrene, p-methylstyrene, o-ethylstyrene, m-ethylstyrene, and p-ethylstyrene; acrylic acid derivative monomers such as methyl acrylate, ethyl acrylate, propyl acrylate, butyl acrylate, octyl acrylate, dodecyl acrylate, stearyl acrylate, behenyl acrylate, 2-ethylhexyl acrylate, dimethylaminoethyl acrylate, diethylaminoethyl acrylate, acrylonitrile, amide acrylate, N-methylacrylamide, N,N-dimethylacrylamide, N-ethylacrylamide, N-(hydroxymethyl)acrylamide, and N-(hydroxyethyl)acrylamide; methacrylic acid derivative monomers such as methyl methacrylate, ethyl methacrylate, propyl methacrylate, butyl methacrylate, octyl methacrylate, dodecyl methacrylate, stearyl methacrylate, behenyl methacrylate, 2-ethylhexyl methacrylate, dimethylaminoethyl methacrylate, diethylaminoethyl methacrylate, methacrylonitrile, amide methacrylate, N-methylmethacrylamide, N,N-dimethylmethacrylamide, and N-(3-dimethylaminopropyl)methacrylamide; olefin monomers such as ethylene, propylene, butylene, butadiene, isoprene, isobutylene, and cyclohexene; halogenated vinyl monomers such as vinyl chloride, vinylidene chloride, vinyl bromide, and vinyl iodide; vinyl ester monomers such as vinyl acetate, vinyl propionate, and vinyl benzoate; vinyl ether monomers such as vinyl methyl ether, vinyl ethyl ether, and vinyl isobutyl ether; and vinyl ketone monomers such as vinyl methyl ketone, vinyl hexyl ketone, and methyl isopropenyl ketone. These monomers can be used alone or optionally in combination of two or more thereof.

As the coloring agent constituting the ink of the present invention, at least a water-insoluble coloring compound represented by Formula (1) is used. The ink can optionally contain another coloring agent that does not impair the solubility or dispersibility of the water-insoluble coloring compound in a medium.

Examples of the coloring agent that can be contained together with the water-insoluble coloring compound represented by Formula (1) in the ink include condensed azo compounds, azo metal complexes, diketopyrrolopyrrole compounds, anthraquinone compounds, quinacridone compounds, naphthol compounds, benzimidazolone compounds, thioindigo compounds, perylene compounds, methine compounds, allylamide compounds, and basic dye lake compounds. Specific examples thereof include, but not limited to, C.I. Pigment Orange 1, 5, 13, 15, 16, 34, 36, 38, 62, 64, 67, 72, and 74; C.I. Pigment Red 2, 3, 4, 5, 6, 7, 12, 16, 17, 23, 31, 32, 41, 48, 48:1, 48:2, 48:3, 48:4, 53:1, 57:1, 81:1, 112, 122, 123, 130, 144, 146, 149, 150, 166, 168, 169, 170, 176, 177, 178, 179, 181, 184, 185, 187, 190, 194, 202, 206, 208, 209, 210, 220, 221, 224, 238, 242, 245, 253, 254, 255, 258, 266, 269, and 282; C.I. Pigment Violet 13, 19, 25, 32, and 50; and various coloring agents classified as derivatives thereof.

The amount of the coloring agent constituting the ink of the present invention may be 1.0 to 30.0 parts by mass, such as 2.0 to 20.0 parts by mass, and even 3.0 to 15.0 parts by mass, based on 100.0 parts by mass of the medium. In such a range, sufficient tinting power can be obtained, and also satisfactory dispersibility of the coloring agent can be achieved.

In the case of using water as the medium of the ink of the present invention, the ink can optionally contain an emulsifier for obtaining satisfactory dispersion stability of the coloring agent. Examples of the emulsifier include, but not limited to, cationic surfactants, anionic surfactants, and nonionic surfactants.

Examples of the cationic surfactant as the emulsifier include dodecyl ammonium chloride, dodecyl ammonium bromide, dodecyl trimethyl ammonium bromide, dodecyl pyridinium chloride, dodecyl pyridinium bromide, and hexadecyl trimethyl ammonium bromide.

Examples of the anionic surfactant as the emulsifier include fatty acid soaps such as sodium stearate and sodium dodecanoate; sodium dodecyl sulfate; sodium dodecyl benzene sulfate; and sodium lauryl sulfate.

Examples of the nonionic surfactant as the emulsifier include dodecyl polyoxyethylene ether, hexadecyl polyoxyethylene ether, nonylphenyl polyoxyethylene ether, lauryl polyoxyethylene ether, sorbitan monooleate polyoxyethylene ether, and monodecanoyl sucrose.

The ink of the present invention can further contain a resin. The type of the resin that can be contained in the ink of the present invention is determined depending on the intended use and is not particularly limited. Examples of the resin include polystyrene resins, styrene copolymers, polyacrylic acid resins, polymethacrylic acid resins, polyacrylate resins, polymethacrylate resins, acrylic acid copolymers, methacrylic acid copolymers, polyester resins, polyvinyl ether resins, polyvinyl methyl ether resins, polyvinyl alcohol resins, polyvinyl butyral resins, polyurethane resins, and polypeptide resins. These resins can be used alone or optionally in combination of two or more thereof.

The dispersing machine used in this step is not particularly limited. For example, a rotation shearing-type homogenizer, a media type dispersing machine such as a ball mill, a sand mill, or an attritor, or a high-pressure counter-collision type dispersing machine can be used.

As described above, the ink of the present invention contains the water-insoluble coloring compound of the present invention having a high affinity to an organic solvent, high brightness and saturation, and spectral reflectance characteristics for a wide color gamut. Accordingly, the present invention can provide an ink having bright red color tone.

The red resist composition for color filter of the present invention will be described.

The ink of the present invention has a bright red color tone and, because of its spectral characteristics, can be suitably used in a resist composition for color filter. In the case of using the ink of the present invention alone, a red resist composition is provided. When the ink of the present invention is mixed with an ink of another color, a resist composition of a secondary color is provided.

The resist composition for color filter of the present invention contains at least a binder resin, a medium, and one or more types of the ink of the present invention.

The resist composition for color filter of the present invention can be obtained, for example, as follows. At least the above-described ink and a binder resin and optionally a polymerizable monomer, a polymerization initiator, and a photoacid generator are gradually added to a medium while stirring for mixing them thoroughly and evenly. Furthermore, the mixture is stably dissolved or fine-dispersed by applying a mechanical shear force with a dispersing machine to obtain a resist composition for color filter of the present invention.

Any binder resin can be used in the resist composition for color filter of the present invention, as long as either a light-irradiating portion or a light-shielding portion is soluble in an organic solvent, an alkali aqueous solution, water, or a commercially available developing solution, in an exposure process of pixel formation. In particular, a resin having a composition that allows developing in water or an alkali aqueous solution can be used from the viewpoint of, for example, workability and waste disposal.

The binder resin is generally obtained by copolymerizing a hydrophilic polymerizable monomer and a hydrophobic polymerizable monomer by a known method at an appropriate mixing ratio. Typical examples of the hydrophilic polymerizable monomer include acrylic acid, methacrylic acid, N-(2-hydroxyethyl)acrylamide, N-vinylpyrrolidone, and polymerizable monomers having ammonium salts. Typical examples of the hydrophobic polymerizable monomer include acrylic acid ester, methacrylic acid ester, vinyl acetate, styrene, and N-vinylcarbazole. Such a binder resin can be used as a negative-type resist, that is, a type where the solubility in a developing solution is decreased by exposure and thereby only the light-shielding portion is removed by developing, in combination with a radical polymerizable monomer having an ethyleny unsaturated group, a cationic polymerizable monomer having an oxirane ring or an oxetane ring, a radical-generating agent, an acid-generating agent, or a base-generating agent.

In addition, for example, an acid-generating agent that generates an acid by exposure can be used in combination with a resin having a quinone diazide group that is cleaved by light to generate a carboxylic acid group or a binder resin having a group that is cleaved by an acid, such as tert-butyl carbonate of polyhydroxystyrene or tetrahydropyranyl ether. Such a binder resin can be used as a positive resist, that is, a type where the solubility in a developing solution is increased by exposure and thereby only the exposure portion is removed by developing.

When the resist composition for color filter of the present invention is a negative-type resist composition, a photopolymerizable monomer having one or more ethyleny unsaturated double bonds is contained in the composition as a polymerizable monomer that is addition-polymerized by exposure. The photopolymerizable monomer is, for example, a compound having at least one addition polymerizable ethyleny unsaturated group in the molecule and having a boiling point of 100° C. or more at ordinary pressure. Examples of the photopolymerizable monomer include monofunctional acrylates such as polyethylene glycol monoacrylate, polyethylene glycol monomethacrylate, polypropylene glycol monoacrylate, polypropylene glycol monomethacrylate, phenoxyethyl acrylate, and phenoxyethyl methacrylate; multi-functional acrylates and methacrylates such as polyethylene glycol diacrylate, polyethylene glycol dimethacrylate, polypropylene glycol diacrylate, polypropylene glycol dimethacrylate, trimethylol ethane triacrylate, trimethylol ethane trimethacrylate, trimethylol propane triacrylate, trimethylol propane trimethacrylate, trimethylol propane diacrylate, trimethylol propane dimethacrylate, neopentyl glycol diacrylate, neopentyl glycol dimethacrylate, pentaerythritol tetraacrylate, pentaerythritol tetramethacrylate, pentaerythritol triacrylate, pentaerythritol trimethacrylate, dipentaerythritol hexaacrylate, dipentaerythritol hexamethacrylate, dipentaerythritol pentaacrylate, dipentaerythritol pentamethacrylate, hexanediol diacrylate, hexanediol dimethacrylate, trimethylol propane tri(acryloyloxypropyl)ether, tri(acryloyloxyethyl)isocyanurate, tri(acryloyloxyethyl)cyanurate, glycerin triacrylate, and glycerin trimethacrylate; and multi-functional acrylates and methacrylates obtained by adding ethylene oxide or propylene oxide to a multi-functional alcohol such as trimethylol propane or glycerin and then performing acrylation or methacrylation. Examples of the photopolymerizable monomer further include urethane acrylates, polyester acrylates, and multi-functional epoxy acrylates and epoxy methacrylates, which are reaction products of epoxy resins and acrylic acid or methacrylic acid. In particular, for example, trimethylol propane triacrylate, trimethylol propane trimethacrylate, pentaerythritol tetraacrylate, pentaerythritol tetramethacrylate, dipentaerythritol hexaacrylate, dipentaerythritol hexamethacrylate, dipentaerythritol pentaacrylate, and dipentaerythritol pentamethacrylate can be used.

The above-mentioned photopolymerizable monomers can be used alone or optionally in combination of two or more thereof.

The content of the photopolymerizable monomer may be 5 to 50% by mass, such as 10 to 40% by mass, based on the mass of the resist composition (the entire solid content) of the present invention. A content of less than 5% by mass may reduce the sensitivity to exposure and the strength of pixels, and a content of larger than 50% by mass may excessively increase the adhesiveness of the resist composition.

When the resist composition for color filter of the present invention is a negative-type resist composition, a photopolymerization initiator is contained in the composition. Examples of the photopolymerization initiator include a vicinal polyketoaldonyl compounds, α-carbonyl compounds, asioin ethers, various quinone compounds, combinations of triallylimidazole dimer and p-aminophenylketone, and trioxadiazole compounds. In particular, 2-benzyl-2-dimethylamino-1-(4-morpholinophenyl)butanone (Trade name: Irgacure 369, manufactured by BASF AG) can be used. When electron rays are used for pixel formation with a color resist of the present invention, the photopolymerization initiator is not essential.

When the resist composition for color filter of the present invention is a positive-type resist composition, the composition may optionally contain a photoacid generator. The photoacid generator may be a known one, and examples thereof include, but not limited to, salts of anions and onium ions such as sulfonium, iodonium, selenium, ammonium, and phosphonium.

Specific examples of the sulfonium ion include triphenylsulfonium, tri-p-tolylsulfonium, tri-o-tolylsulfonium, tris(4-methoxyphenyl)sulfonium, 1-naphthyldiphenylsulfonium, diphenylphenacylsulfonium, phenylmethylbenzylsulfonium, 4-hydroxyphenylmethylbenzylsulfonium, dimethyl phenacylsulfonium, and phenacyltetrahydrothiophenium.

Specific examples of the iodonium ion include diphenyliodonium, di-p-tolyliodonium, bis(4-dodecylphenyl)iodonium, bis(4-methoxyphenyl)iodonium, and (4-octyloxyphenyl)phenyliodonium.

Specific examples of the selenium ion include triarylseleniums (e.g., triphenylselenium, tri-p-tolylselenium, tri-o-tolylselenium, tris(4-methoxyphenyl)selenium, 1-naphtyldiphenylselenium, tris(4-fluorophenyl)selenium, tri-1-naphthylselenium, and tri-2-naphthylselenium).

Specific examples of the ammonium ion include tetraalkylammoniums (e.g., tetramethylammonium, ethyltrimethylammonium, diethyldimethylammonium, triethylmethylammonium, tetraethylammonium, trimethyl-n-propylammonium, trimethylisopropylammonium, trimethyl-n-butylammonium, and trimethylisobutylammonium).

Specific examples of the phosphonium ion include tetraphenylphosphonium, tetra-p-tolylphosphonium, tetrakis(2-methoxyphenyl)phosphonium, triphenylbenzylphosphonium, triphenylphenacylphosphonium, triphenylmethylphosphonium, triethylbenzylphosphonium, and tetraethylphosphonium.

Specific examples of the anion include, but not limited to, perhalogenic acid ions such as ClO₄⁻ and BrO₄⁻; halogenated sulfonate ions such as FSO₃⁻ and ClSO₃⁻; sulfate ions such as CH₃SO₄⁻, CF₃SO₄⁻, and HSO₄⁻; carbonate ions such as HCO₃⁻ and CH₃CO₃⁻; aluminate ions such as AlCl₄⁻ and AlF₄⁻; a hexafluorobismuthic acid ion; carboxylate ions such as CH₃COO⁻, CF₃COO—, C₆H₅COO⁻, CH₃C₆H₄COO⁻, C₆F₅COO⁻, and CF₃C₆H₄COO⁻; arylborate ions such as B(C₆H₅)₄⁻ and CH₃CH₂CH₂CH₂B(C₆H₅)₃⁻; a thiocyanate ion; and a nitrate ion.

In the resist composition for color filter of the present invention, examples of the medium for dissolving or dispersing the ink, the binder resin, and optionally the photopolymerizable monomer, photopolymerization initiator, and photoacid generator include water and various organic solvents. Examples of the organic solvent include cyclohexanone, ethyl cellosolve acetate, butyl cellosolve acetate, 1-methoxy-2-propyl acetate, diethylene glycol dimethyl ether, ethyl benzene, 1,2,4-trichlorobenzene, ethylene glycol diethyl ether, xylene, ethyl cellosolve, methyl-n-amyl ketone, propylene glycol monomethyl ether, toluene, methyl ethyl ketone, ethyl acetate, methanol, ethanol, isopropanol, butanol, methyl isobutyl ketone, and petroleum solvents. These organic solvents can be used alone or in combination of two or more thereof. The medium of the resist composition for color filter of the present invention may be the same as or different from the medium used for the ink as long as the dispersibility of the coloring agent in the ink is not impaired.

In a color filter where two or more types of pixels having different spectral characteristics are arranged so as to be adjacent to each other, the filter can have high brightness and saturation and good color tone by using the resist composition containing the ink of the present invention for at least pixels constituting one color among the plurality of colors (e.g., red, green, and blue). In order to further obtain desired spectral characteristics, the composition can contain another dye for tone adjustment. The additionally used dye is not particularly limited, and examples of the dye include C.I. Solvent Blue 14, 24, 25, 26, 34, 37, 38, 39, 42, 43, 44, 45, 48, 52, 53, 55, 59, 67, and 70; C.I. Solvent Red 8, 27, 35, 36, 37, 38, 39, 40, 49, 58, 60, 65, 69, 81, 83:1, 86, 89, 91, 92, 97, 99, 100, 109, 118, 119, 122, 127, and 218; and C.I. Solvent Yellow 1, 2, 3, 13, 14, 19, 21, 22, 29, 36, 37, 38, 39, 40, 42, 43, 44, 45, 47, 62, 63, 71, 76, 79, 81, 82, 83:1, 85, 86, 88, and 151.

The resist composition for color filter of the present invention may optionally contain an ultraviolet absorber and a silane coupling agent for improving adhesiveness with a glass substrate in the process of producing the filter, in addition to the above-described additives.

The dispersing machine used in this step is not particularly limited. For example, a rotation shearing-type homogenizer, a media type dispersing machine such as a ball mill, a sand mill, or an attritor, or a high-pressure counter-collision type dispersing machine can be used.

As described above, the resist composition for color filter of the present invention contains the ink having a bright red color tone of the present invention and can thereby have a bright red color tone.

The thermal transfer recording sheet of the present invention will now be described.

The ink of the present invention has a bright red color tone and can be suitably used in a thermal transfer recording sheet because of its spectral characteristics. In the case of using the ink of the present invention alone, a red thermal transfer recording sheet is provided. In the case of mixing the ink of the present invention with an ink of another color, a thermal transfer recording sheet having a secondary color is provided.

The thermal transfer recording sheet of the present invention includes a base material and a coloring material layer that is composed of at least the ink of the present invention on the base material.

The thermal transfer recording sheet of the present invention can be obtained by, for example, as follows. At least a coloring agent containing a water-insoluble coloring compound represented by Formula (1) and a binder resin and optionally, for example, a surfactant and a wax are gradually added to a medium while stirring for mixing them thoroughly and evenly. Furthermore, the composition is stably dissolved or dispersed in a fine particle state with a mechanical shear force applied by a dispersing machine to obtain an ink of the present invention. Subsequently, the ink is applied to a base film serving as the base material and dried to produce a thermal transfer recording sheet of the present invention, but the present invention is not limited to the thermal transfer recording sheet produced by this method.

Various resins can be used as the binder resin for the thermal transfer recording sheet of the present invention. Specific examples thereof include resins soluble in aqueous solutions, such as cellulose resins, polyacrylic acid resins, starch resins, and epoxy resins, and resins soluble in organic solvents, such as polyacrylate resins, polymethacrylate resins, polystyrene resins, polycarbonate resins, polyether sulfone resins, polyvinyl butyral resins, ethyl cellulose resins, acetyl cellulose resins, polyester resins, AS resins, and phenoxy resins. These resins can be used alone or optionally in combination of two or more thereof.

In the method of producing the thermal transfer recording sheet, a medium that is used as the medium of the ink can be similarly used. Specific examples of the medium include water and organic solvents. Examples of the organic solvent include alcohols such as methanol, ethanol, isopropanol, and isobutanol; cellosolves such as methyl cellosolve and ethyl cellosolve; aromatic hydrocarbons such as toluene, xylene, and chlorobenzene; esters such as ethyl acetate and butyl acetate; ketones such as acetone, methyl ethyl ketone, methyl isobutyl ketone, and cyclohexanone; halogenated hydrocarbons such as methylene chloride, chloroform, and trichloroethylene; ethers such as tetrahydrofuran and dioxane; N,N-dimethylformamide; and N-methylpyrrolidone. The organic solvents can be used alone or optionally in combination of two or more thereof.

The thermal transfer recording sheet of the present invention can have high brightness and saturation and good color tone by using at least the water-insoluble coloring compound represented by Formula (1) as a coloring agent. In order to further achieve desired spectral characteristics, the sheet can contain another dye for tone adjustment. The additionally used dye is not particularly limited as long as brightness, saturation, and light resistance of the thermal transfer recording sheet of the present invention are not highly affected, and examples of the dye include C.I. Solvent Red 8, 27, 35, 36, 37, 38, 39, 40, 49, 58, 60, 65, 69, 81, 83:1, 86, 89, 91, 92, 97, 99, 100, 109, 118, 119, 122, 127, and 218; C.I. Disperse Red 1, 59, 60, 73, 135, 146, and 167; and C.I. Disperse Violet 26.

The mass ratio of the binder resin to the coloring agent (binder resin : coloring agent) can be in a range of 1:2 to 2:1, from the viewpoint of a transferring property.

The thermal transfer recording sheet of the present invention can contain a surfactant for providing a sufficient lubricating property during heating the thermal head (during printing). Examples of the surfactant that can be contained in the thermal transfer recording sheet include cationic surfactants, anionic surfactants, and nonionic surfactants.

Examples of the cationic surfactant include dodecyl ammonium chloride, dodecyl ammonium bromide, dodecyl trimethyl ammonium bromide, dodecyl pyridinium chloride, dodecyl pyridinium bromide, and hexadecyl trimethyl ammonium bromide.

Examples of the anionic surfactant include fatty acid soaps such as sodium stearate and sodium dodecanoate; sodium dodecyl sulfate; sodium dodecyl benzene sulfate; and sodium lauryl sulfate.

Examples of the nonionic surfactant include dodecyl polyoxyethylene ether, hexadecyl polyoxyethylene ether, nonylphenyl polyoxyethylene ether, lauryl polyoxyethylene ether, sorbitan monooleate polyoxyethylene ether, and monodecanoyl sucrose.

The thermal transfer recording sheet of the present invention can contain a wax for providing a sufficient lubricating property during non-heating the thermal head. Examples of the wax that can be contained in the thermal transfer recording sheet include, but not limited to, polyethylene waxes, paraffin waxes, and fatty acid ester waxes.

The thermal transfer recording sheet of the present invention may optionally contain an ultraviolet absorber, an antiseptic, an antioxidant, an anti-static agent, or a viscosity modifier, in addition to the above-described additives.

The base film serving as the base material for the thermal transfer recording sheet of the present invention is not particularly limited. For example, thin paper such as condenser paper or glassine paper or a plastic film of polyester, polycarbonate, polyamide, polyimide, or polyaramide can be used as a base material having high heat resistance; and a polyethylene terephthalate film can be used as a base material having high mechanical strength, solvent resistance, and cost performance. The thickness of the base material can be 3 to 50 μm, from the viewpoint of a transferring property.

The thermal transfer recording sheet of the present invention can have a layer of a lubricant, a heat-resistant fine particles having a high lubricating property, and a thermal resin such as a binding agent on the opposite side of the coloring material layer, for increasing the heat resistance and the mobility of the thermal head. Examples of the lubricant include, but not limited to, amino-modified silicone compounds and carboxy-modified silicone compounds. Examples of the heat-resistant fine particles include, but not limited to, fine particles of silica, and examples of the binding agent include, but not limited to, acrylic resins.

Examples of the dispersing machine that is used in the dispersing step include, but not limited to, a rotation shearing-type homogenizer, a media type dispersing machine such as a ball mill, a sand mill, and an attritor, and a high-pressure counter-collision type dispersing machine.

The method of applying the ink composition to the base film is not particularly limited, and examples thereof include methods using a bar coater, a gravure coater, a reverse roll coater, a rod coater, or an air doctor coater. The application amount of the ink composition can be controlled so that the coloring material layer after drying has a thickness of 0.1 to 5 μm, from the viewpoint of a transferring property.

The thermal transfer recording sheet of the present invention may be heated by any method without particular limitation. For example, not only a thermal head, which is usually used, but also infrared rays or laser can be used. Alternatively, an electrification exothermic film that generates heat by electrifying the base film itself may be used as an electrification dye transfer sheet.

As described above, the thermal transfer recording sheet of the present invention contains the ink having a bright red color tone of the present invention and thereby can be provided as a thermal transfer recording sheet having a bright red color tone.

### EXAMPLES

The present invention will be described in more detail by the following Examples and Comparative Examples, but is not limited to these Examples. Note that “part(s)” and “%” in Examples and Comparative Examples are based on mass unless otherwise specified. The identification of obtained reaction products was performed by a plurality of analytic methods using apparatuses: a ¹H NMR spectrometer (ECA-400, manufactured by JEOL Ltd.), a LC/TOF mass spectrometer (LC/MSD TOF, manufactured by Agilent Technologies, Inc.), and a UV/Vis spectrophotometer (UV-36000 spectrophotometer, manufactured by Shimadzu Corporation). Ionization in the LC/TOF MS was performed by electrospray ionization (ESI).

A water-insoluble coloring compound represented by Formula (1) was produced by the method described below.

### Synthesis Example 1

### Production of Water-insoluble Coloring Compound (5)

3-Acetylamino-2,4,6-trimethylaniline (7.3 g) and Compound A (7.4 g) shown in the above-described synthesis scheme were heated at 150° C. for 3 hours for reaction in sulfolane (20 mL) in the presence of zinc chloride (4.1 g). This solution was cooled and was then poured into 50 mL of a 2 mol/L hydrochloric acid solution. The precipitated crystals were separated by filtration, washed with water, and then crystallized from acetone to yield the water-insoluble coloring compound (5).

¹H-NMR analysis, LC/TOF MS analysis, and UV/Vis spectroscopic analysis of the water-insoluble coloring compound (5) were performed with the above-mentioned analytical apparatuses. The analytical results are shown below.

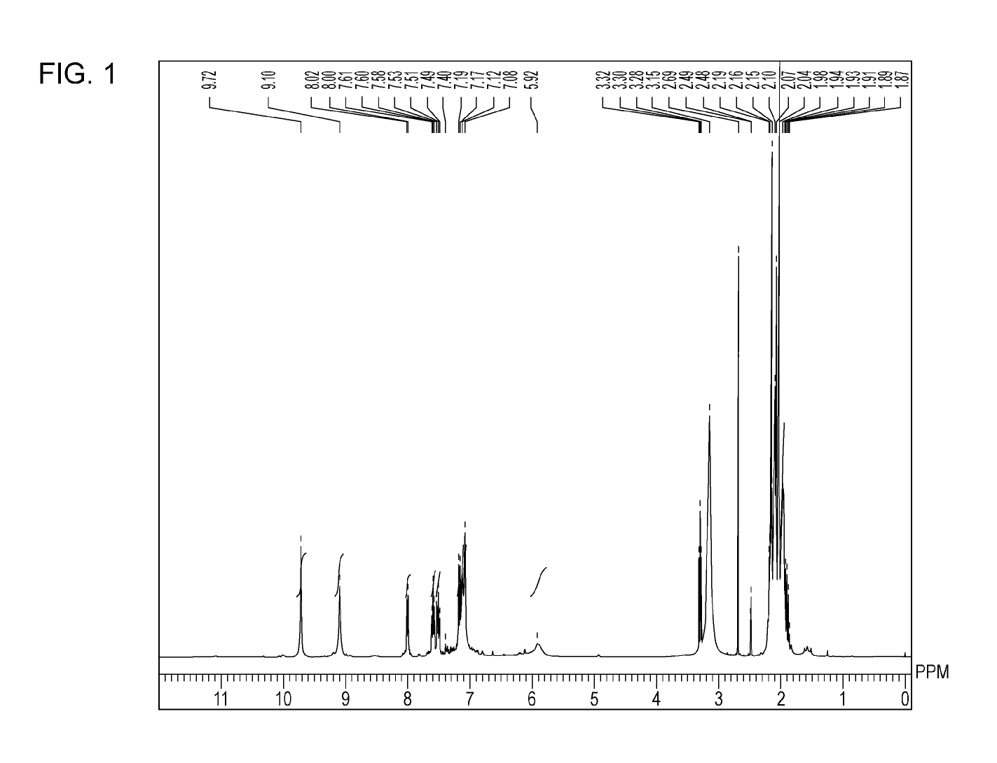

### Analytical Results of Water-insoluble Coloring Compound (5)

[1] Result of ¹H-NMR (400 MHz, DMSO-d₆, 80° C.) (see FIG. 1): δ [ppm]=9.72 (s, 2H), 9.10 (s, 2H), 8.01 (d, 1H, J=7.63 Hz), 7.60 (t, 1H, J=7.25 Hz), 7.51 (t, 1H, J=7.63 Hz), 7.18-7.08 (m, 7H), 5.92 (br, 1H), 2.16-1.98 (m, 24H).

[2] Mass spectrometry (ESI-TOF): m/z=715.2696 (M-H)⁻.

[3] Result of UV/Vis spectroscopic analysis: λₘₐₓ=530 nm (CH₃OH: 2.5×10⁻⁵ mol/L).

All of water-insoluble coloring compound (5) obtained above and water-insoluble coloring compounds (1) to (4) and (6) to (25) produced below had a solubility in water of less than 1% as mass percentage and were thus water-insoluble compounds.

### Synthesis Example 2

### Production of Water-insoluble Coloring Compound (6)

Water-insoluble coloring compound (6) was prepared by the same method as in Synthesis Example 1 except that 3-propionylamino-2,4,6-trimethylaniline was used, instead of 3-acetylamino-2,4,6-trimethylaniline, in an amount of 1.3 times the number of moles of the 3-acetylamino-2,4,6-trimethylaniline in Synthesis Example 1.

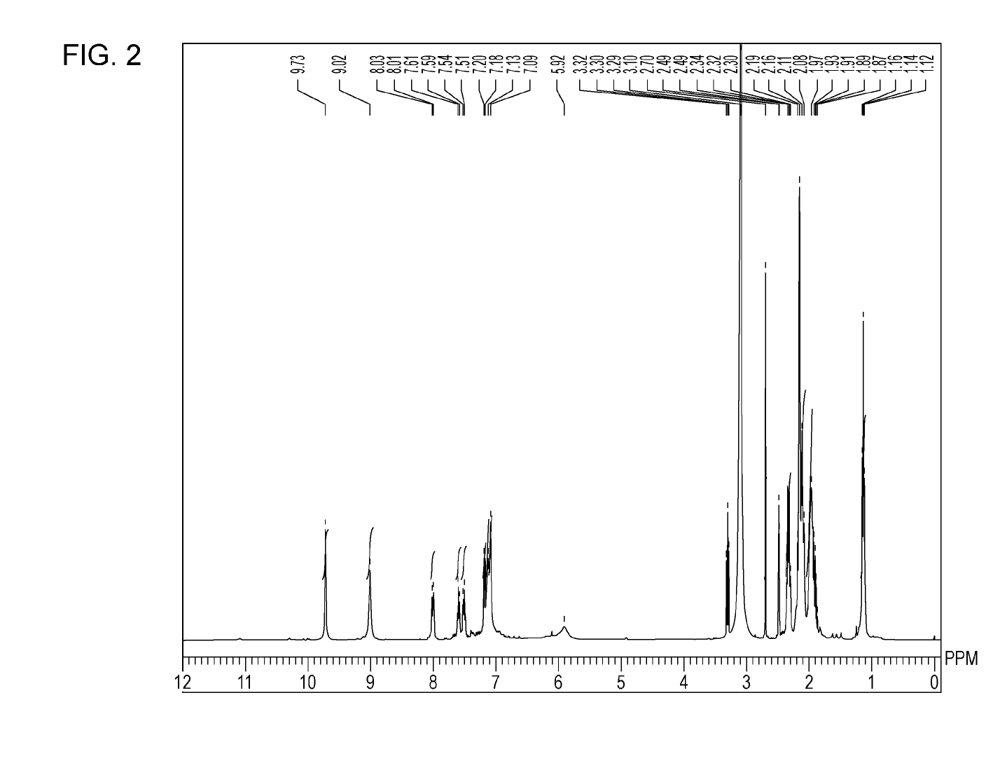

### Analytical Results of Water-insoluble Coloring Compound (6)

[1] Result of ¹H NMR (400 MHz, DMSO-d₆, 80° C.) (see FIG. 2): δ [ppm]=9.73 (s, 2H), 9.02 (s, 2H), 8.02 (d, 1H, J=7.63 Hz), 7.60 (t, 1H, J=7.63 Hz), 7.53 (t, 1H, J=8.39 Hz), 7.19-7.09 (m, 7H), 5.92 (br, 1H), 2.32 (t, 4H, J=7.63 Hz), 2.16-1.97 (m, 16H), 1.14 (t, 6H, J=7.63 Hz).

[2] Mass spectrometry (ESI-TOF): m/z=743.2976(M-H)⁻.

[3] Result of UV/Vis spectroscopic analysis: λₘₐₓ=530 nm (CH₃OH: 2.5×10⁻⁵ mol/L).

### Synthesis Example 3

### Production of Water-insoluble Coloring Compound (7)

Water-insoluble coloring compound (7) was prepared by the same method as in Synthesis Example 1 except that 3-butylamino-2,4,6-trimethylaniline was used, instead of 3-acetylamino-2,4,6-trimethylaniline, in an amount of 1.5 times the number of moles of the 3-acetylamino-2,4,6-trimethylaniline in Synthesis Example 1.

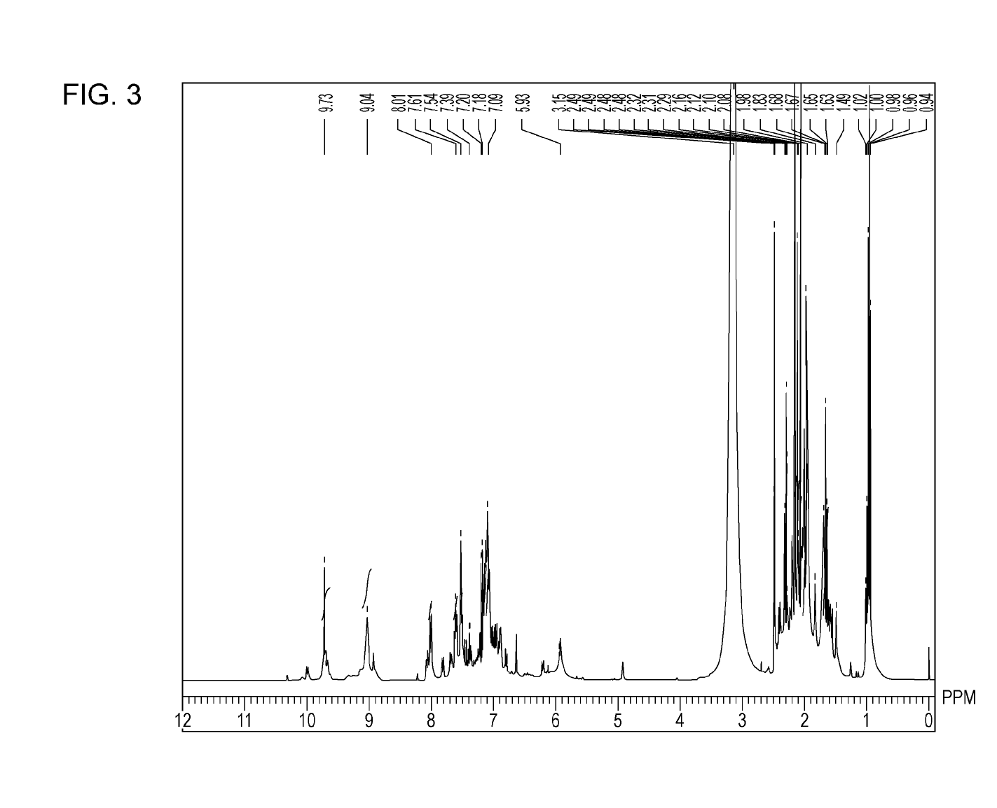

### Analytical Results of Water-insoluble Coloring Compound (7)

[1] Result of ¹H-NMR (400 MHz, DMSO-d₆, 80° C.) (see FIG. 3): δ [ppm]=9.73 (s, 2H), 9.04 (s, 2H), 8.01 (d, 1H, J=7.63 Hz), 7.61 (t, 1H, J =7.63 Hz), 7.54 (t, 1H, J=8.39 Hz), 7.19-7.09 (m, 7H), 5.93 (br, 1H), 2.31 (t, 4H, J=7.25 Hz), 2.16-1.98 (m, 18H), 1.66 (dd, 6H, J=14.9, 7.25 Hz), 0.96 (t, 6H, J=7.25 Hz).

[2] Mass spectrometry (ESI-TOF): m/z=771.3306 (M-H).

[3] Result of UV/Vis spectroscopic analysis: λₘₐₓ=530 nm (CH₃OH: 2.5×10⁻⁵ mol/L).

### Synthesis Example 4

### Production of Water-insoluble Coloring Compound (8)

Water-insoluble coloring compound (8) was prepared by the same method as in Synthesis Example 1 except that 3-isobutylamino-2,4,6-trimethylaniline was used, instead of 3-acetylamino-2,4,6-trimethylaniline, in the same number of moles of the 3-acetylamino-2,4,6-trimethylaniline in Synthesis Example 1 and that the amount of sulfolane was changed to twice that in Synthesis Example 1.

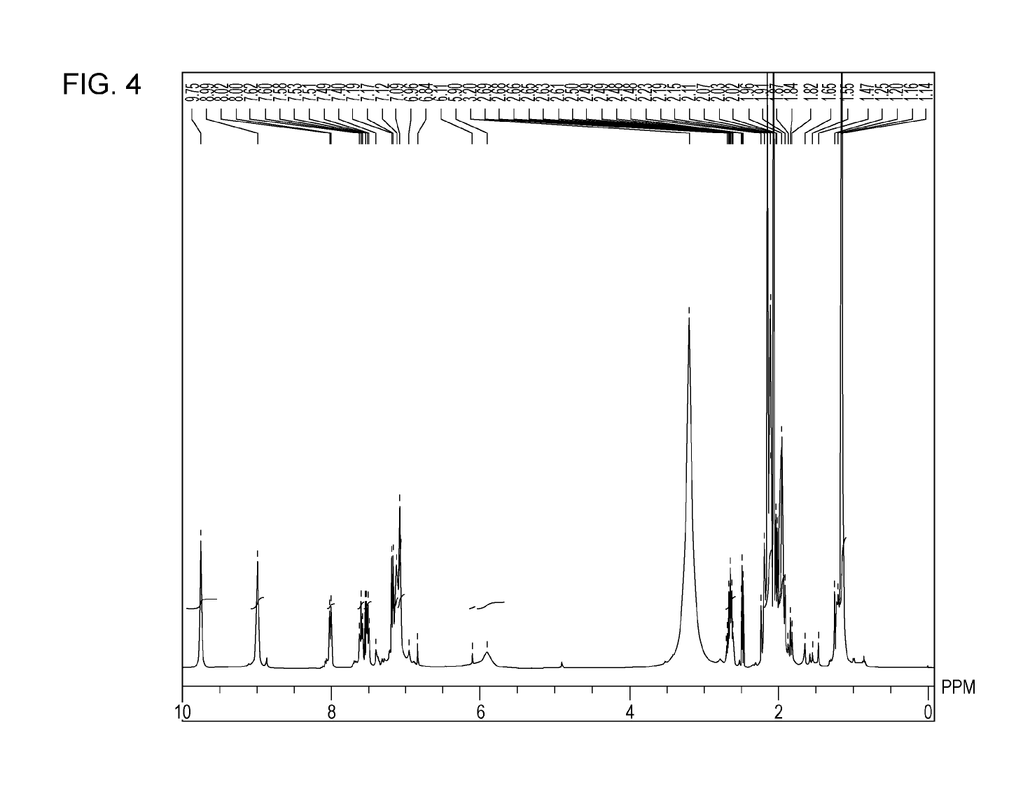

### Analytical Results of Water-insoluble Coloring Compound (8)

[1] Result of ¹H-NMR (400 MHz, DMSO-d₆, 80° C.) (see FIG. 4): δ [ppm]=9.75 (s, 2H), 8.99 (s, 2H), 8.01 (d, 1H, J=8.39 Hz), 7.60 (t, 1H, J=7.63 Hz), 7.51 (t, 1H, J=8.01 Hz), 7.18-7.09 (m, 7H), 5.90 (br, 1H), 2.65 (td, 2H, J=13.4, 6.36 Hz), 2.13 (m, 11H) , 1.96 (m, 6H) , 1.15 (m, 13H).

[2] Mass spectrometry (ESI-TOF): m/z=771.3295 (M-H)⁻.

[3] Result of UV/Vis spectroscopic analysis: λₘₐₓ=530 nm (CH₃OH: 2.5×10⁻⁵ mol/L).

### Synthesis Example 5

### Production of Water-insoluble Coloring Compound (10)

Water-insoluble coloring compound (10) was prepared by the same method as in Synthesis Example 1 except that 3-benzoylamino-2,4,6-trimethylaniline was used, instead of 3-acetylamino-2,4,6-trimethylaniline, in an amount of 1.8 times the number of moles of the 3-acetylamino-2,4,6-trimethylaniline in Synthesis Example 1.

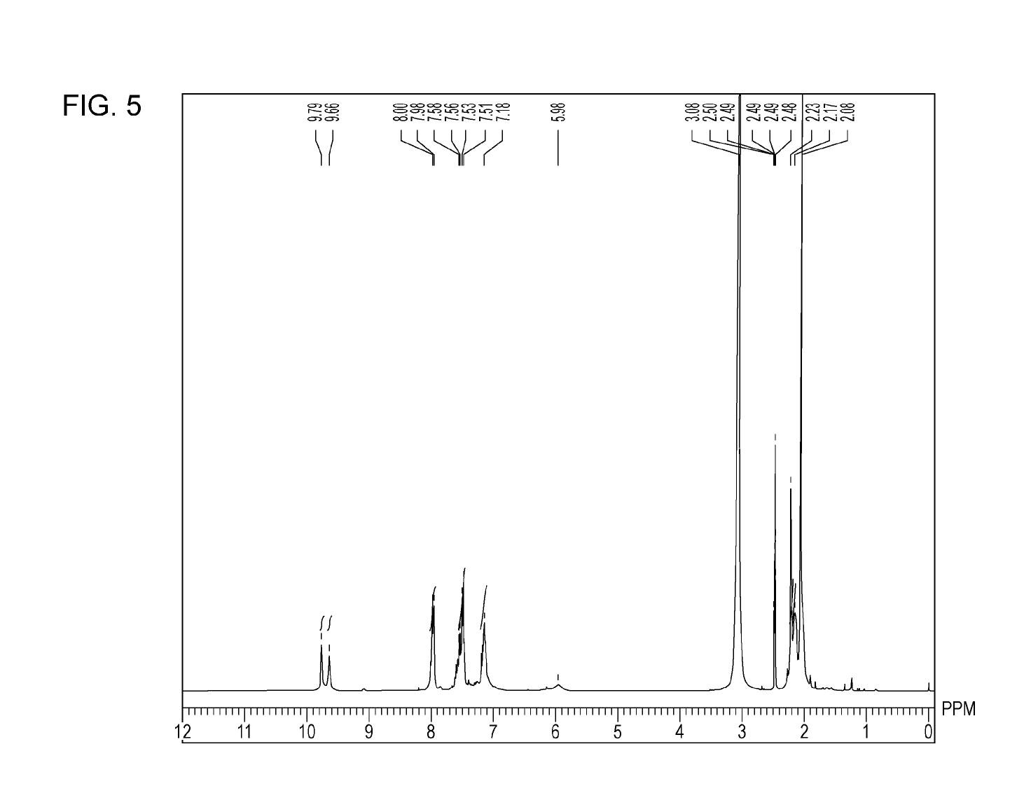

### Analytical Results of Water-insoluble Coloring Compound (10)

[1] Result of ¹H-NMR (400 MHz, DMSO-d₆, 80° C.) (see FIG. 5): δ [ppm] =9.79 (s, 2H), 9.66 (s, 2H), 7.99 (d, 6H, J=7.63 Hz), 7.58-7.51 (m, 8H), 7.18 (m, 6H), 7.18-7.09 (m, 7H), 5.98 (br, 1H), 2.23-2.08 (m, 18H).

[2] Mass spectrometry (ESI-TOF): m/z=839.2973 (M-H)⁻.

[3] Result of UV/Vis spectroscopic analysis: λₘₐₓ=530 nm (CH₃OH: 2.5×10⁻⁵ mol/L).

### Synthesis Example 6

### Production of Water-insoluble Coloring Compound (24)

Water-insoluble coloring compound (24) was prepared by the same method as in Synthesis Example 1 except that 3-(2-heptylundecanoylamino)-2,4,6-trimethylaniline was used, instead of 3-acetylamino-2,4,6-trimethylaniline, in an amount of twice the number of moles of the 3-acetylamino-2,4,6-trimethylaniline in Synthesis Example 1.

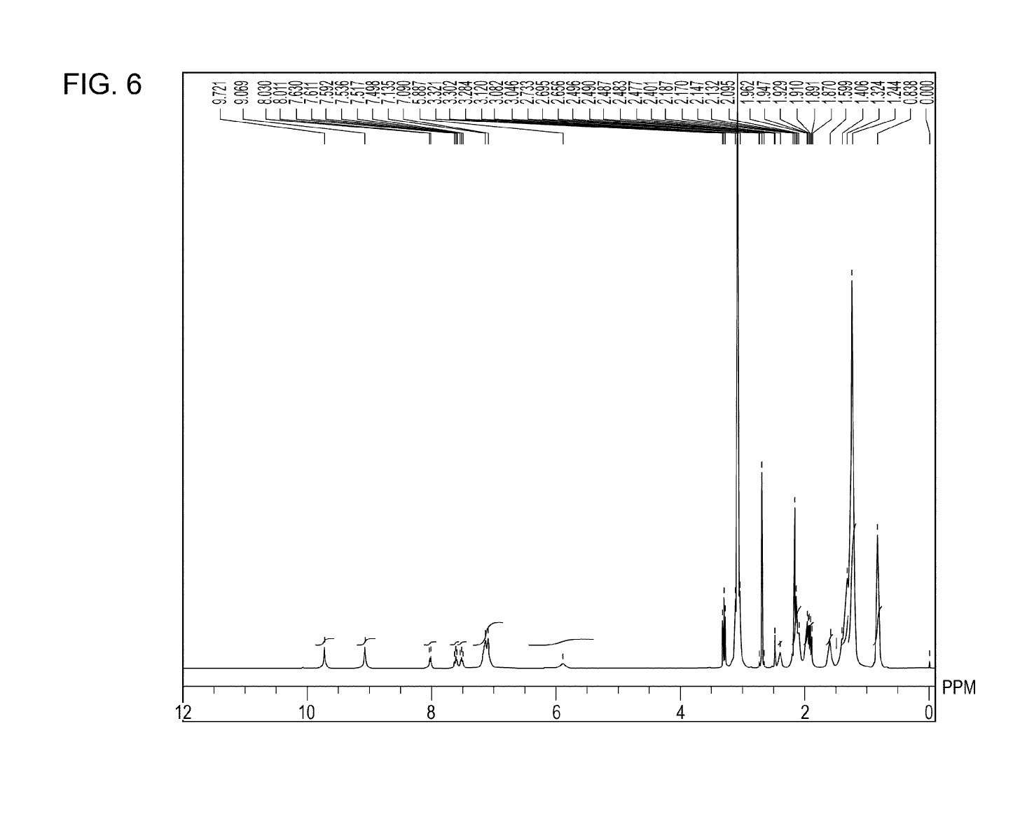

### Analytical Results of Water-insoluble Coloring Compound (24)

[1] Result of ¹H-NMR (400 MHz, DMSO-d₆, 80° C.) (see FIG. 6): δ [ppm]=9.72 (s, 2H), 9.07 (s, 2H), 8.02 (d, 1H, J=7.63 Hz), 7.61 (t, 1H, J=7.63 Hz), 7.52 (t, 1H, J=7.63 Hz), 7.11 (m, 7H), 5.89 (br, 2H), 3.30 (t, 2H, J=7.25 Hz), 2.69 (s, 3H), 2.40 (s, 1H), 2.19-2.10 (m, 12H), 1.92 (m, 7H), 1.60 (s, 3H), 1.41 (s, 1H), 1.32 (s, 9H), 1.24 (s, 38H), 0.84 (s, 12H).

[2] Mass spectrometry (ESI-TOF): m/z=1165.7665 (M+H)⁺.

[3] Result of UV/Vis spectroscopic analysis: λₘₐₓ=531 nm (CH₃OH: 2.5×10⁻⁵ mol/L).

### Synthesis Example 7

### Production of Water-insoluble Coloring Compound (25)

Water-insoluble coloring compound (25) was prepared by the same method as in Synthesis Example 1 except that 3-(2-(1,3,3-trimethylbutyl)-5,7,7-trimethyl)octanoylamino-2,4,6-trimethylaniline was used, instead of 3-acetylamino-2,4,6-trimethylaniline, in an amount of twice the number of moles of the 3-acetylamino-2,4,6-trimethylaniline in Synthesis Example 1.

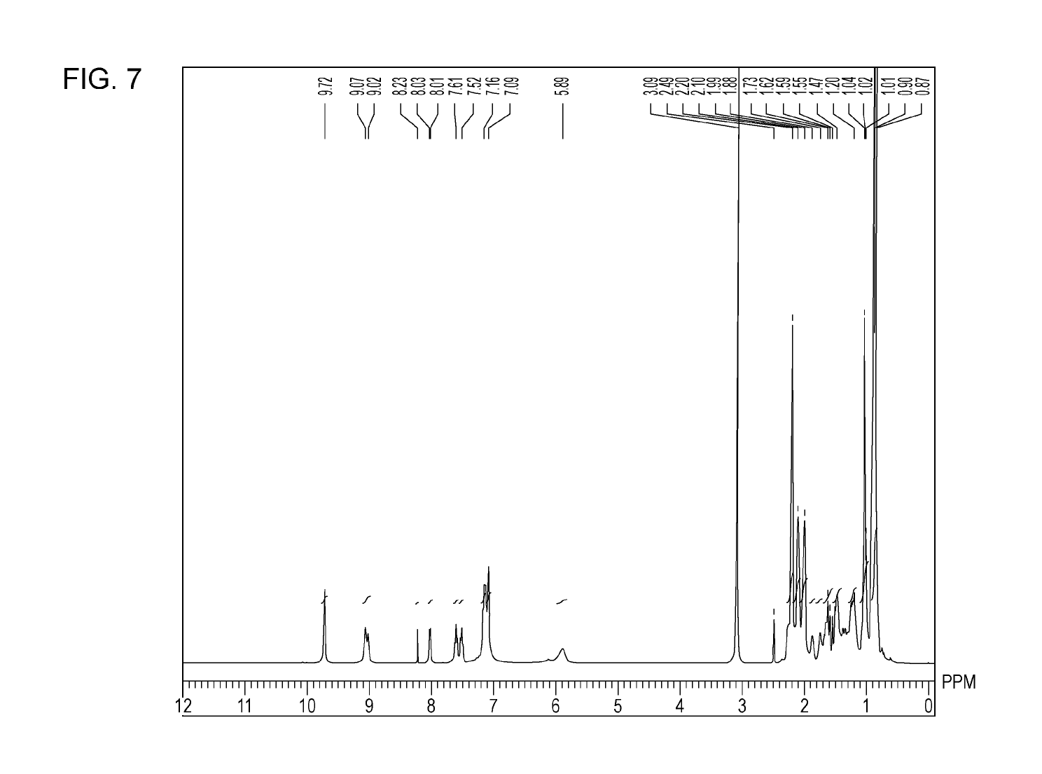

### Analytical Results of Water-insoluble Coloring Compound (25)

[1] Result of ¹H-NMR (400 MHz, DMSO-d₆, 80° C.) (see FIG. 7): δ [ppm]=9.72 (s, 2H), 9.04 (s, 2H), 8.02 (d, 1H, J=7.63 Hz), 7.61 (t, 1H, J=7.63 Hz), 7.52 (t, 1H, J=8.01 Hz), 7.16-7.09 (m, 7H), 5.89 (br, 1H), 2.20-1.99 (m, 18H), 1.99-1.58 (m, 8H), 1.47-1.20 (m, 12H), 1.02-0.87 (m, 48H).

[2] Mass spectrometry (ESI-TOF): m/z=1165.7878 (M+H)⁺.

[3] Result of UV/Vis spectroscopic analysis: λₘₐₓ=531 nm (CH₃OH: 2.5×10⁻⁵ mol/L).

### Synthesis Examples of Other Water-insoluble Coloring Compounds

Water-insoluble coloring compounds (9) and (11) to (23) shown in Table 1 were synthesized by the method according to Synthesis Examples 1 to 7. The synthesized water-insoluble coloring compounds were confirmed by H NMR analysis, LC/TOF MS analysis, and UV/Vis spectroscopic analysis using the above-mentioned apparatuses.

In Table 1, “nC₁₇H₁₅” represents a normal steary group, and “*” represents a binding site.

TABLE 1
Structure of coloring compound represented by Formula (1)

| Compound No. | R₁ | R₂ | R₃ | R₄ | R₅ |
|---|---|---|---|---|---|
| (5) | CH₃ | CH₃CONH | CH₃ | H | CH₃ |
| (6) | CH₃ | CH₃CH₂CONH | CH₃ | H | CH₃ |
| (7) | CH₃ | CH₃CH₂CH₂CONH | CH₃ | H | CH₃ |
| (8) | CH₃ | (CH₃)₂CHCONH | CH₃ | H | CH₃ |
| (9) | CH₃ | 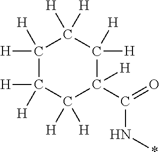 [SMILES: *NC(=O)C1CCCCC1] | CH₃ | H | CH₃ |
| (10) | CH₃ | 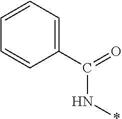 [SMILES: *NC(=O)c1ccccc1] | CH₃ | H | CH₃ |
| (11) | CH₃ | CH₃CONH | CH₃ | CH₃CONH | CH₃ |
| (12) | CH₃ | 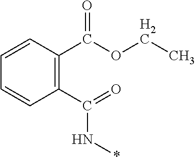 [SMILES: *NC(=O)c1ccccc1C(=O)OCC] | CH₃ | H | CH₃ |
| (13) | CH₃ | CH₃CONH | CH₃ | CH₃CONH | CH₃ |
| (14) | CH₃CH₂ | CH₃CONH | H | H | CH₃CH₂ |
| (15) | CH₃CH₂ | CH₃CONH | H | H | CH₃CH₂ |
| (16) | (CH₃)₂CH | CH₃CONH | H | H | (CH₃)₂CH |
| (17) | CH₃ | CH₃CONH | CH₃ | CH₃CONH | CH₃ |
| (18) | CH₃ | CH₃CONH | CH₃CH₂O | H | CH₃ |
| (19) | CH₃ | CH₃CONH | 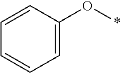 [SMILES: *Oc1ccccc1] | H | CH₃ |
| (20) | CH₃ | 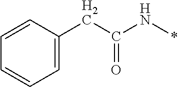 [SMILES: *NC(=O)Cc1ccccc1] | CH₃ | H | CH₃ |
| (21) | CH₃ | 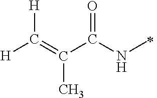 | CH₃ | H | CH₃ |
| (22) | CH₃ | 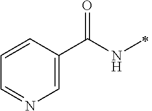 | CH₃ | H | CH₃ |
| (23) | CH₃ | 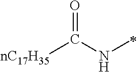 | CH₃ | H | CH₃ |
| (24) | CH₃ | 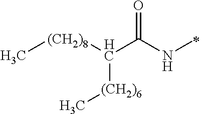 | CH₃ | H | CH₃ |
| (25) | CH₃ | 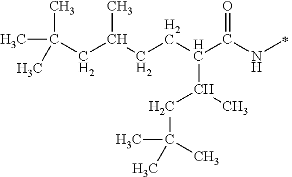 | CH₃ | H | CH₃ |
| Compound No. | R₆ | R₇ | R₈ | R₉ | R₁₀ |
| (5) | CH₃ | CH₃CONH | CH₃ | H | CH₃ |
| (6) | CH₃ | CH₃CH₂CONH | CH₃ | H | CH₃ |
| (7) | CH₃ | CH₃CH₂CH₂CONH | CH₃ | H | CH₃ |
| (8) | CH₃ | (CH₃)₂CHCONH | CH₃ | H | CH₃ |
| (9) | CH₃ | 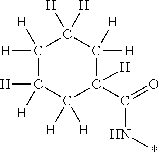 [SMILES: *NC(=O)C1CCCCC1] | CH₃ | H | CH₃ |
| (10) | CH₃ | 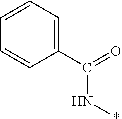 [SMILES: *NC(=O)c1ccccc1] | CH₃ | H | CH₃ |
| (11) | CH₃ | CH₃CONH | CH₃ | CH₃CONH | CH₃ |
| (12) | CH₃ | 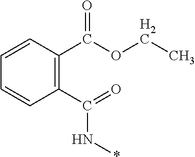 [SMILES: *NC(=O)c1ccccc1C(=O)OCC] | CH₃ | H | CH₃ |
| (13) | CH₃ | CH₃CONH | CH₃ | H | CH₃ |
| (14) | CH₃CH₂ | CH₃CONH | H | H | CH₃CH₂ |
| (15) | CH₃ | CH₃CONH | H | H | CH₃ |
| (16) | CH₃ | CH₃CONH | H | H | CH₃ |
| (17) | CH₃ | CH₃CONH | H | CH₃CONH | CH₃ |
| (18) | CH₃ | CH₃CONH | CH₃CH₂O | H | CH₃ |
| (19) | CH₃ | CH₃CONH | 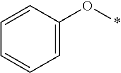 [SMILES: *Oc1ccccc1] | H | CH₃ |
| (20) | CH₃ | 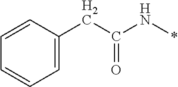 [SMILES: *NC(=O)Cc1ccccc1] | CH₃ | H | CH₃ |
| (21) | CH₃ | 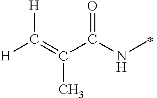 | CH₃ | H | CH₃ |
| (22) | CH₃ | 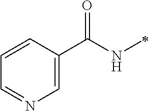 | CH₃ | H | CH₃ |
| (23) | CH₃ | 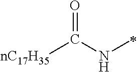 | CH₃ | H | CH₃ |
| (24) | CH₃ | 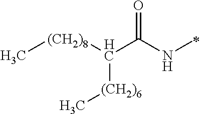 | CH₃ | H | CH₃ |
| (25) | CH₃ | 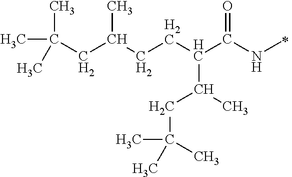 | CH₃ | H | CH₃ |

### Comparative Coloring Compounds (1) to (3)

As coloring compounds for comparison, Comparative Compounds (1) to (3) were prepared.

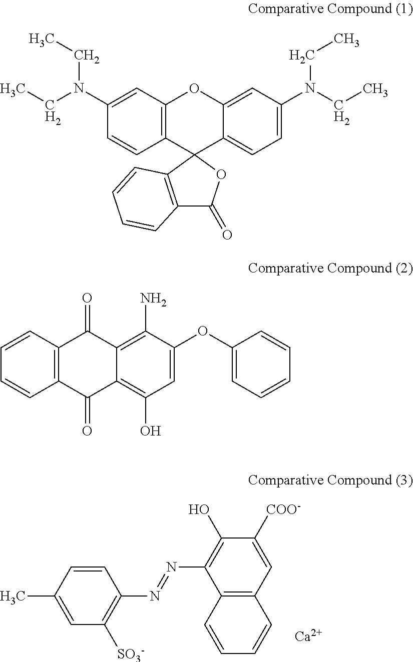

### Production of Ink

Inks of the present invention and comparative inks were produced by the method described below.

### Production Example of Ink (1)

Seventeen parts of water-insoluble coloring compound (5) of the present invention and 120 parts of styrene were mixed and charged in an attritor (manufactured by Mitsui Mining Co., Ltd.), followed by operation for 1 hour to prepare ink (1) of the present invention. Production Examples of inks (2) to (21)

Inks (2) to (21) were prepared by the same procedure as in Production Example of ink (1) except that water-insoluble coloring compounds (6) to (25) were used instead of water-insoluble coloring compound (5) in Production Example of ink (1).

### Production Examples of Comparative Inks (1) to (3)

Comparative inks (1) to (3) were prepared by the same procedure as in Production Example of ink (1) except that comparative compounds (1) to (3) were used instead of water-insoluble coloring compound (5) in Production Example of ink (1).

### Evaluation

### Evaluation of Solubility of Compound in Solvent

Thirty milligrams of each of water-insoluble coloring compounds (5) to (25) and comparative compounds (1) to (3) was dissolved in 0.7 mL of methanol, chloroform, ethyl acetate, or toluene at room temperature. The solubility in each solvent was visually evaluated by the following criteria:

A: completely dissolved (the solubility is excellent),

B: slightly remaining suspended solids (the solubility is good), and

C: not dissolved at all (the solubility is poor).

### Measurement of color gamut

Inks (1) to (21) and comparative inks (1) to (3) were each applied to paper for hiding ratio measurement by bar coating (Bar No. 4, 6, 8, 10, 12, 14, 16, 18, and 20) and were air-dried overnight to produce image samples. Chromaticity (L*, a*, and b*) in the L*a*b* color system of each image sample was measured with a reflection densitometer SpectroLino (manufactured by Gretag Macbeth AG).

### Color Tone Evaluation

Color tone was evaluated as follows.

A larger expansion of chromaticity in the magenta color gamut at the same L* refers to a better magenta color tone. Accordingly, the color tone was evaluated by the a* and b* values at an L* of 60. The a* and b* values at an L* of 60 were determined by interpolation of L*, a*, and b* obtained from each image sample. The color tone was evaluated by the following criteria:

A: a* is 70 or more and b* is 0 or less (the color tone is excellent),

B: a* is 70 or more and b* is higher than 0, or a* is less than 70 and b* is 0 or less (the color tone is good), and

C: a* is less than 70 and b* is higher than 0 (the color tone is poor).

### Saturation Evaluation

Saturation was evaluated as follows.

A larger saturation c* at the same coloring agent amount per unit area refers to a better saturation. The saturation c* of the image sample produced by bar coating (Bar No. 10) was used for evaluation.

The c* is calculated by {(a*)²+(b*)²}^{1/2}. The saturation was evaluated by the following criteria:

A: c* is 80 or more (the saturation is excellent),

B: c* is 70 or more and less than 80 (the saturation is good), and

C: c* is less than 70 (the saturation is poor).

### Light Resistance Evaluation

The sample of which L* value was the closest to 60 among the image samples produced by bar coating was charged in Atlas Weather-Ometer Ci4000 (manufactured by Toyo Seiki Seisaku-Sho, Ltd.) and was subjected to an exposure test for 36 hours. The measurement conditions were as follows:

### Black Panel: 50° C.,

### Chamber: 40° C.,

### Relative Humidity: 70%, and

### Irradiance (340 nm): 0.39 W/m².

Before and after the exposure test, chromaticity (L*, a*, and b*) in the L*a*b* color system of the sample was measured with a reflection densitometer SpectroLino (manufactured by Gretag Macbeth AG). The color difference (ΔE) was calculated from the measured values of color properties by the following expression:

Color difference (ΔE)=[{(a* before the test)−(a* after the test)}²+{(b* before the test)−(b* after the test)}²+{(L* before the test)−(L* after the test)}²]^{1/2}

When the ΔE after 36 hours was less than 10, the light resistance was evaluated as good.

Table 2 shows the results of each evaluation of the water-insoluble coloring compounds and comparative compounds.

TABLE 2
Results of evaluation of compounds

| | Solubility | | | | | a*/b*/color tone | c*/saturation | |
|---|---|---|---|---|---|---|---|---|
| Compound | | | Ethyl | | | evaluation at L* = | evaluation with | |
| No. | Methanol | Chloroform | acetate | Toluene | Ink No. | 60 | bar coater No. 10 | ΔE (36 h) |
| (5) | A | A | A | A | (1) | 80.8/−16.2/A | 85.2/A | 3.2 |
| (6) | A | A | A | A | (2) | 80.5/−16.0/A | 85.6/A | 3.3 |
| (7) | A | A | A | A | (3) | 81.4/−16.3/A | 85.1/A | 3.2 |
| (8) | A | A | A | A | (4) | 81.6/−15.8/A | 83.5/A | 2.9 |
| (9) | A | A | A | A | (5) | 80.9/−15.9/A | 85.0/A | 3.5 |
| (10) | A | A | A | A | (6) | 78.9/−15.9/A | 82.5/A | 3.8 |
| (11) | A | A | A | A | (7) | 80.1/−16.3/A | 84.5/A | 2.3 |
| (12) | A | A | A | A | (8) | 80.8/−15.8/A | 83.9/A | 3.5 |
| (13) | A | A | A | A | (9) | 80.2/−16.0/A | 84.0/A | 3.1 |
| (14) | A | A | A | A | (10) | 79.9/−15.7/A | 83.5/A | 5.2 |
| (15) | A | A | A | A | (11) | 80.5/−15.7/A | 84.4/A | 5.0 |
| (16) | A | A | A | A | (12) | 79.2/−16.4/A | 85.2/A | 5.8 |
| (17) | A | A | A | A | (13) | 80.6/−16.5/A | 84.6/A | 4.9 |
| (18) | A | A | A | A | (14) | 81.2/−15.5/A | 84.6/A | 3.9 |
| (19) | A | A | A | A | (15) | 81.0/−15.9/A | 81.5/A | 4.5 |
| (20) | A | A | A | A | (16) | 79.8/−15.2/A | 82.2/A | 5.4 |
| (21) | A | A | A | A | (17) | 78.6/−16.0/A | 82.1/A | 7.8 |
| (22) | A | A | A | A | (18) | 77.9/−16.5/A | 81.7/A | 5.0 |
| (23) | A | A | A | A | (19) | 79.6/−17.2/A | 80.9/A | 3.8 |
| (24) | A | A | A | A | (20) | 80.0/−16.7/A | 81.1/A | 3.5 |
| (25) | A | A | A | A | (21) | 79.9/−17.1/A | 80.9/A | 4.1 |
| Comparative | A | A | B | B | Comparative | 86.2/−34.3/A | 88.9/A | 15.8 |
| compound (1) | | | | | ink (1) | | | |
| Comparative | C | C | C | C | Comparative | 66.1/−1.7/B | 76.9/B | 12.3 |
| compound (2) | | | | | ink (2) | | | |
| Comparative | C | C | C | C | Comparative | 64.0/−9.6/B | 67.2/C | 1.5 |
| compound (3) | | | | | ink (3) | | | |

As obvious from Table 2, comparative compounds (1) to (3) do not satisfy any of the requirements in the solubility in solvent, color tone, saturation, and light resistance. On the other hand, the water-insoluble coloring compounds of the present invention have high solubility in solvent, good color tone, high saturation, and high light resistance.

### Production of Red Resist Composition

### Production Example of Color Filter (1)

Twelve parts of water-insoluble coloring compound (7) of the present invention and 120 parts of cyclohexanone were mixed and charged in an attritor (manufactured by Mitsui Mining Co., Ltd.), followed by operation for 1 hour to prepare ink (21) of the present invention.

Twenty-two parts of ink (21) was gradually added to a solution of 6.7 parts of an acrylic copolymer composition, 1.3 parts of dipentaerythritol pentaacrylate, and 0.4 parts of 2-benzyl-2-dimethylamino-1-(4-morpholinophenyl)-butanone (photopolymerization initiator) in 96 parts of cyclohexanone at room temperature for 3 hours with stirring. This was filtrated with a filter of 1.5 μm to prepare red resist composition (1) for color filter of the present invention.

Red resist composition (1) for color filter was spin-coated on a glass substrate and was then dried at 90° C. for 3 min, and the entire surface was then exposed and post-cured at 180° C. to produce color filter (1).

### Production Example of Color Filters (2) and (3)

Color filters (2) and (3) were produced by the same procedure as in Production Example of color filter (1) except that red resist compositions (2) and (3) for color filter respectively containing water-insoluble coloring compounds (10) and (25) were used, respectively, instead of water-insoluble coloring compound (7) in Production Example of color filter (1).

### Production Example of Color Filter (4)

Color filter (4) was produced by the same procedure as in Production Example of color filter (3) except that 0.2 parts of 1-butanol was added to red resist composition (3) for color filter.

### Production Example of Comparative Color Filter (1)

Comparative color filter (1) was produced by the same procedure as in Production Example of color filter (1) except that comparative compound (1) was used instead of water-insoluble coloring compound (7).

### Production of Thermal Transfer Recording Sheet

### Production Example of Thermal Transfer Recording Sheet (1)

Five parts of a polyvinyl butyral resin (Denka 3000-K, manufactured by Denki Kagaku Kogyo K.K.) was gradually added to a solution mixture of 13.5 parts of methyl ethyl ketone/45 parts of toluene containing water-insoluble coloring compound (5) of the present invention with stirring to prepare ink (22) of the present invention.

Ink (22) was applied onto a polyethylene terephthalate film (Lumirror, manufactured by Toray Industries, Inc.) having a thickness of 4.5 μm and was dried to produce thermal transfer recording sheet (1).

### Production Examples of Thermal Transfer Recording Sheets (2) and (3)

Thermal transfer recording sheets (2) and (3) were produced by the same procedure as in Production Example of thermal transfer recording sheet (1) except that water-insoluble coloring compounds (6) and (20) were respectively used instead of water-insoluble coloring compound (5).

### Production Example of Thermal Transfer Recording Sheet (4)

Thermal transfer recording sheet (4) was produced by the same procedure as in Production Example of thermal transfer recording sheet (3) except that 1.4 parts of 1-butanol was further added.

### Production Example of Comparative Thermal Transfer Recording Sheet (1)

Comparative thermal transfer recording sheet (1) was produced by the same procedure as in Production Example of thermal transfer recording sheet (1) except that comparative compound (2) was used instead of water-insoluble coloring compound (5).

### Evaluation

### Measurement of Color Gamut of Color Filter

Color filter (1) and comparative color filter (1) were each placed on paper for hiding ratio measurement. Chromaticity (L*, a*, and b*) in the L*a*b* color system was measured with a reflection densitometer SpectroLino (manufactured by Gretag Macbeth AG). Saturation (c*) was calculated by the above-mentioned computational expression from the measured values of color properties.

### Measurement of Color Gamut of Transferred Image

Thermal transfer recording sheet (1) and comparative thermal transfer recording sheet (1) were each cut and pasted to the magenta portion of an ink cassette for SELPHY CP710 (manufactured by CANON KABUSHIKI KAISHA), and solid images of a single magenta color were formed on exclusive printing paper with SELPHY CP710 (manufactured by CANON KABUSHIKI KAISHA) as transferred image (1) and comparative transferred image (1). Chromaticity (L*, a*, and b*) in the L*a*b* color system of each transferred image was measured with a reflection densitometer SpectroLino (manufactured by Gretag Macbeth AG).

### Color Tone Evaluation

Color tone was evaluated as follows.

A larger expansion of chromaticity in the magenta color gamut at the same L* refers to a better magenta color tone. Accordingly, the color tone was evaluated by the a* and b* values at an L* of 50. The sample of an L* of 50 was produced by controlling the temperature in the image formation with SELPHY CP710. The color tone was evaluated by the following criteria:

A: a* is 70 or more and b* is 30 or less (the color tone is excellent),

B: a* is 70 or more and b* is higher than 30, or a* is less than 70 and b* is 30 or less (the color tone is good), and

C: a* is less than 70 and b* is higher than (the color tone is poor).

### Saturation Evaluation

A larger saturation c* at the same coloring agent amount per unit area refers to a better saturation. The color filters and transferred images at a coloring agent amount of 6.5 mg for 25 cm² (5 cm×5 cm) of each transferred image were evaluated using the c* value by the following criteria:

A: c* is 80 or more (the saturation is excellent),

B: c* is 70 or more and less than 80 (the saturation is good), and

C: c* is less than 70 (the saturation is poor).

### Light Resistance Evaluation

Each of the samples used in the color tone evaluation was charged in Atlas Weather-Ometer Ci4000 (manufactured by Toyo Seiki Seisaku-Sho, Ltd.) and was subjected to an exposure test for 36 hours. The measurement conditions were as follows:

### Black Panel: 50° C.,

### Chamber: 40° C.,

### Relative Humidity: 70%, and

### Irradiance (340 nm): 0.39 W/m².

Before and after the exposure test, chromaticity (L*, a*, and b*) in the L*a*b* color system of each sample was measured with a reflection densitometer SpectroLino (manufactured by Gretag Macbeth AG). The color difference (ΔE) was calculated from the measured values of color properties by the above-mentioned expression.

When the ΔE after 36 hours was less than 10, the light resistance was evaluated as good.

TABLE 3
Evaluation results

| | | a*/b*/color | c*/ | |
|---|---|---|---|---|
| | | tone evaluation | saturation | ΔE |
| | Compound No. | at L* = 50 | evaluation | (36 h) |
| Color filter (1) | (7) | 81.0/−2.9/A | 87.4/A | 5.0 |
| Color filter (2) | (10) | 79.0/−3.9/A | 85.3/A | 6.1 |
| Color filter (3) | (25) | 82.2/−3.4/A | 88.2/A | 6.2 |
| Color filter (4) | (25) | 83.3/−4.2/A | 89.7/A | 5.8 |
| Thermal transfer | (5) | 85.2/−5.2/A | 84.4/A | 2.5 |
| recording sheet | | | | |
| (1) | | | | |
| Thermal transfer | (6) | 84.8/−5.0/A | 85.0/A | 2.4 |
| recording sheet | | | | |
| (2) | | | | |
| Thermal transfer | (20) | 84.2/−3.5/A | 86.6/A | 3.6 |
| recording sheet | | | | |
| (3) | | | | |
| Thermal transfer | (20) | 85.8/−5.5/A | 87.4/A | 3.6 |
| recording sheet | | | | |
| (4) | | | | |
| Comparative | Comparative | 88.2/−12.4/A | 84.3/A | 17.8 |
| color filter (1) | compound (1) | | | |
| Comparative | Comparative | 74.8/10.1/B | 70.2/B | 11.5 |
| thermal transfer | compound (2) | | | |
| recording sheet | | | | |
| (1) | | | | |

As obvious from Table 3, the color filters produced by the red resist composition for color filter of the present invention and the transferred image produced by the thermal transfer recording sheet of the present invention had high color tone and saturation, spectral reflectance characteristics for wide color gamuts, and high light resistance, compared to the color filter produced by the corresponding comparative resist composition and the transferred image produced by the comparative thermal transfer recording sheet.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2011-059555, filed Mar. 17, 2011, which is hereby incorporated by reference herein in its entirety.

### Industrial Applicability

The water-insoluble coloring compound represented by Formula (1) has high solubility in solvent, good color tone and saturation, spectral reflectance characteristics for a wide color gamut, and a high light resistance. Accordingly, the water-insoluble coloring compound can be used in inks for color filters and inks for thermal transfer sheets and also can be suitably used as a dye for optical recording media, a printing ink, a paint, and a writing material ink.

1. A water-insoluble coloring compound represented by Formula (1): 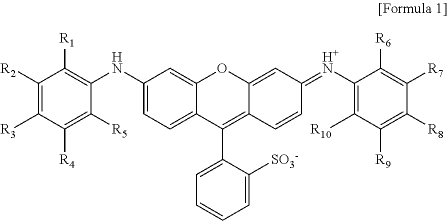
[wherein, R₁, R₅, R₆, and R₁₀ each independently represent an alkyl group; R₃ and R₈ each independently represent a hydrogen atom, an alkyl group, an alkoxy group, or an aryloxy group; and R₂, R₄, R₇, and R₉ each independently represent a hydrogen atom or an acylamino group represented by Formula (2), and at least one of R₂, R₄, R₇, and R₉ is an acylamino group represented by Formula (2): 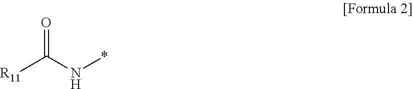
(wherein, R₁₁ represents an alkyl group, a cycloalkyl group, an aryl group, an arylalkyl group, an alkenyl group, or a heterocyclic group; and * represents a binding site)].

2. The water-insoluble coloring compound according to claim 1, wherein R₁₁ in Formula (2) is an alkyl group or an aryl group.

3. The water-insoluble coloring compound according to claim 1, wherein R₁₁ in Formula (2) is a linear alkyl group.

4. The water-insoluble coloring compound according to claim 1, wherein, in Formula (1), the substituents R₁, R₂, R₃, R₄, and R₅ are the same as the substituents R₆, R₇, R₈, R₉, and R₁₀, respectively.

5. An ink comprising the water-insoluble coloring compound according to claim 1.

6. A resist composition for color filter, comprising the ink according to claim 5.

7. A thermal transfer recording sheet comprising a base material and a color material layer formed of a composition containing the ink according to claim 5 on the base material.
